(19) United States
(12) Patent Application Publication
(10) Pub. No.: US 2009/0030417 A1
(43) Pub. Date: Jan. 29, 2009

(54) Drill guide for anterior cruciate ligament reconstruction operation

(76) Inventor: Toshiaki Takahashi, Ehime (JP)

Correspondence Address: WENDEROTH, LIND & PONACK, L.L.P., 2033 K STREET N. W., SUITE 800, WASHINGTON, DC, 20006-1021, US

(21) Appl. No.: 12/219,060

(22) Filed: Jul. 15, 2008

(30) Foreign Application Priority Data
Jul. 17, 2007 (JP) 2007-186165

(51) Int. Cl. A61B 17/58

(52) U.S. Cl. 606/96

(57) ABSTRACT

A drill guide for an anterior cruciate ligament reconstruction operation is provided. The drill guide allows a transplantation hole on a tibial bone and a transplantation hole on a femoral bone to be properly positioned without relying upon the experience and sense of the operator. The drill guide for an anterior cruciate ligament reconstruction operation includes a guide member regulating a movement of a drill guide in a direction perpendicular to an axis of the same, a pointing member having a tip part located in a position on an imaginary line extending from an axis of the guide member, and a light beam radiating member radiating a light beam traveling along the imaginary line extending from the axis of the guide member.

### BACKGROUND OF THE INVENTION

1. Field of the Invention

The present invention relates to a medical device used in a treatment for reconstructing a damaged anterior cruciate ligament.

2. Description of the Related Art

An injury of an anterior cruciate ligament (which may be hereinafter abbreviated as “ACL”) mostly occurs during a sports activity, and the frequency of occurrence of such injuries is on the increase reflecting the increasing sporting population. As described in Non-Patent Document 1, transplantation of hamstrings or the like is carried out to cure an injury of an anterior cruciate ligament. Holes for transplantation must be formed in the femoral bone and the tibial bone by drilling them in order to transplant the tendon graft.

Patent Documents 1 and 2 also describe inventions of apparatus to be used for an ACL reconstruction operation.

Patent Document 1: JP-A-5-1845219

Patent Document 2: JP-T-2006-500182

Non-Patent Document 1: Yagi M, Kuroda R, Nagamune K, Kurosaka M., “Double-bundle ACL reconstruction can improve rotational stability.” Clin Orthop Relat Res. 2007; 454:100-7.

An ACL reconstruction operation is carried out while observing the injured part with an endoscope inserted in the joint. Holes for transplantation are formed in the femoral bone and the tibial bone by drilling them while adjusting the position of the drill with a drill guide. The holes for transplantation must be accurately formed in positions where the anterior cruciate ligament should be located. Drill guides in practical use are equipped with a pointing member pointing one position in the direction in which the drill proceeds. The pointing member is positioned under observation through an endoscope such that it abuts on the position of the tibial bone where the hole is to be formed. Thus, the tibial bone side transplantation hole can be formed in a proper position.

However, once drilling is started, the direction of the drill cannot be changed, and the drill can only proceed in the same direction. The transplantation hole on the femoral bone side is formed in the position of the femoral bone where the drill reaches after proceeding as thus described. Therefore, when drilling is started, both of the positions of the tibial bone side transplantation hole and the femoral bone side transplantation hole must have been properly determined. However, drill guides according to the related art are not provided with measures to assist the positioning of a transplantation hole on a femoral bone, and operations have been performed relying upon the experience of doctors. Neither Patent Document 1 nor Patent Document 2 describes any measures to assist the positioning of a transplantation hole on a femoral bone.

### SUMMARY OF THE INVENTION

It is an object of the invention to provide a drill guide for an ACL reconstruction operation which allows a transplantation hole on a tibial bone and a transplantation hole on a femoral bone to be properly positioned without relying upon the experience and sense of an operator.

In order to solve the above-described problem, a drill guide for an ACL reconstruction operation according to the invention includes a guide member regulating a movement of a drill blade in a direction perpendicular to an axis of the same, a pointing member having a tip part located in a position on an imaginary line extending from an axis of the guide member, and a light beam radiating member radiating a light beam traveling along the imaginary line extending from the axis of the guide member.

The drill guide may include a pointing member supporting arm supporting the pointing member and a mirror provided at a tip part of the pointing member supporting arm, and the pointing member supporting arm may include a water supplying part.

The drill guide for an ACL reconstruction operation according to the invention is advantageous in that a transplantation hole on a femoral bone and a transportation hole on a tibial bone can be properly positioned under observation through an endoscope using the pointing member and the light beam traveling along the imaginary line extending from the axis of the guide member.

### BRIEF DESCRIPTION OF THE DRAWINGS

FIG. 1 is a plan view of a drill guide for an ACL reconstruction operation; and

FIG. 2 is a conceptual diagram showing a state of a drill guide for an ACL reconstruction operation in use.

### DETAILED DESCRIPTION OF THE INVENTION

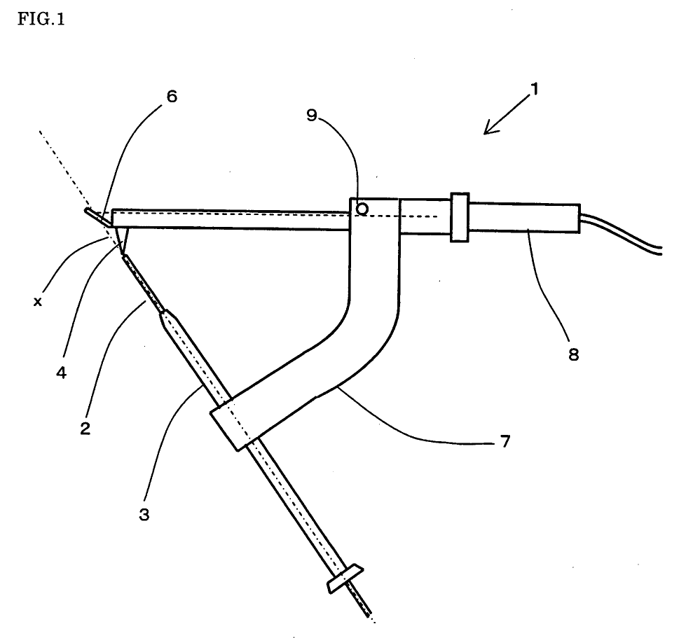

An embodiment of the invention will now be described with reference to the drawings. FIG. 1 is a plan view of a drill guide for an ACL reconstruction operation.

A drill guide 1 for an ACL reconstruction operation includes a guide member 3 regulating a movement of a drill blade 2 in a direction perpendicular to an axis of the same, a pointing member 4 having a tip part located on a position on an imaginary line x extending from an axis of a guide member 3, and a light beam radiating member 6 radiating a light beam traveling along the imaginary line x extending from the axis of the guide member 3.

The guide member 3 may be formed from a metal tube. The member has a hole through which the drill blade 2 can penetrate. Therefore, although the drill blade 2 can move in axial directions thereof in the guide member 3, the movement of the drill blade 2 in lateral directions or directions perpendicular to the axis of the drill blade is limited.

The pointing member 4 is provided in the form of a protrusion in the vicinity of a tip part of a pointing member supporting arm 5. The tip of the pointing member 4 in the form of a protrusion points a position near a tip part of the guide member 3 on the imaginary line x extending from the axis of the guide member 3. The pointing member 4 has the function of positioning a transplantation hole on a tibial bone. The guide member 3 and the pointing member supporting arm 5 are connected to each other through a grip section 7.

The light beam radiating member 6 radiates a light beam traveling along the imaginary line x extending from the axis of the guide member 3, and the member has the function of positioning a transplantation hole on a femoral bone. In this embodiment, the light beam radiating member is formed by a laser light generating device 8 provided at the base of the pointing member supporting arm 5 and a mirror 6. The laser light generating device 8 maybe similar to those in pointers used for presentation. The mirror 6 is provided at the tip part of the pointing member supporting arm 5. Laser light emitted by the laser light generating device 8 travels along the pointing member supporting arm 5. The light is reflected by the mirror 6, and it then travels along the imaginary line x extending from the axis of the guide member 3. With such a configuration, the size of the part of the device inserted in the knee joint can be kept small.

Instead of the light beam radiating member shown in FIG. 1, laser light may be emitted by a light-emitting member such as a light-emitting diode provided at the tip part of the pointing member supporting arm 5. Alternatively, light may be introduced from an external light source through an optical fiber.

The pointing member supporting arm 5 is provided with a water inlet port 9. A tube (not shown) may be attached to the water inlet port 9 to supply water into the optical path. By filling the optical path with water as thus described, air-water interfaces can be eliminated from the optical path to eliminate refraction at air-water interfaces, whereby a proper optical path can be provided.

A description will now be made on operations of the drill guide for an ACL reconstruction operation and a method for using the same. The ACL reconstruction operation has already become popular, and many articles have been published on the same including Non-Patent Document 1. An operation performed using the drill guide for ACL reconstruction according to the invention is mostly based on such known ACL reconstruction operation. Therefore, the description will omit details of regions where the operation according to the invention overlaps the known ACL reconstruction operation.

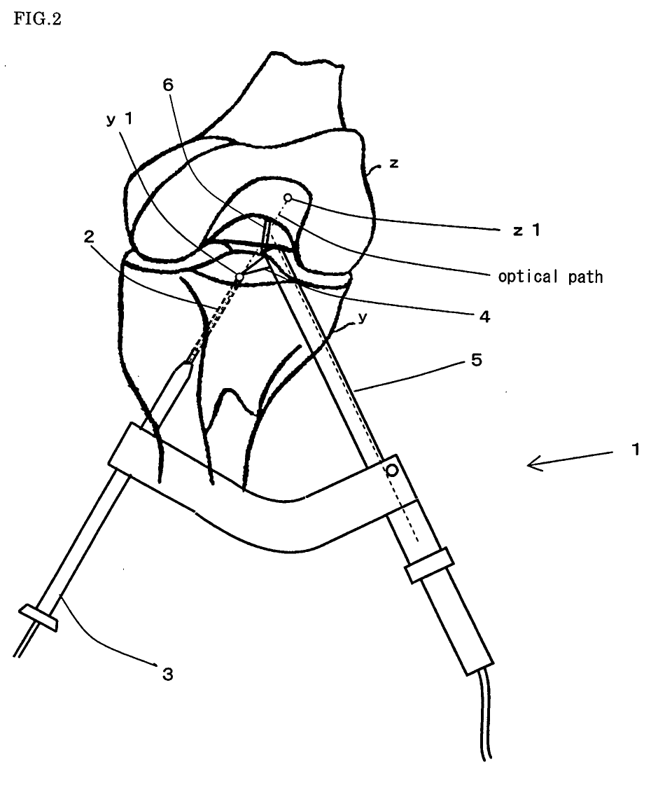

FIG. 2 is a conceptual illustration showing a state of the drill guide for an ACL reconstruction operation in use. The patient lies on his or her back on an operating table with the knee to be operated bent upward. An endoscope (not shown) is inserted into the knee joint to allow observation of the interior of the knee joint. In addition to the hole for inserting the endoscope, another hole is provided to insert the pointing member supporting arm 5 into the knee joint.

Under observation through the endoscope, the pointing member 4 is made to abut on a position y1 of a tibial bone y where a transplantation hole is to be formed. Thus, the position of the exit where the drill is to appear in the knee joint after cutting a hole in the tibial bone y is determined.

The positioning of a transplantation hole on a femoral bone z is performed with the pointing member 4 kept abutting on the position y1 where the tibial bone side transplantation hole is to be formed. The direction of the laser light which has entered the knee joint after traveling from the laser light generating device 8 along the pointing member supporting arm 5 is changed by the mirror 6, and the light then travels along a path in which the drill is to proceed to cut a hole. Under observation through the endoscope, the direction of the drill guide 1 for an ACL reconstruction operation is adjusted such that the laser light impinges upon a position z1 of the femoral bone z where a transplantation hole is to be formed.

When the positioning of the tibial bone side transplantation hole and the femoral bone side transplantation hole has been completed as thus described, holes are cut using the drill with the positioning kept unchanged. The drill blade 2 is inserted into the guide member 3, and the drill blade is made to proceed along the guide member 3 to cut holes. The drill blade enters the knee joint through the position where the pointing member 4 is abut on and reaches the femoral bone in the position identified by the laser light. Thus, a tibial bone side transplantation hole and a femoral bone side transplantation hole are properly formed.

When the tibial bone side transplantation hole and the femoral bone side transplantation hole are formed, a tendon graft is implanted according to a known method of operation. The description of such steps is omitted. Recently, transplantation of a double bundle graft is proposed as an approach to more precise reconstruction of an anterior cruciate ligament. In this case, it is necessary to form two series of tibial bone side transplantation holes and femoral bone side transplantation holes. With the drill guide for an ACL reconstruction operation according to the invention, such steps can be simply and accurately carried out.

As thus described, the drill guide for an ACL reconstruction operation makes it possible to form a tibial bone side transplantation hole and a femoral bone side transplantation hole in proper positions. The positioning of the transplantation holes requires not so much experience. Since the invention is mostly based on methods of operation which have already become popular, those skilled in such techniques have substantially nothing to be newly learned to perform an operation using the drill guide for ACL reconstruction.

What is claimed is:

1. A drill guide for an anterior cruciate ligament reconstruction operation, comprising:
a guide member regulating a movement of a drill blade in a direction perpendicular to an axis of the same;
a pointing member having a tip part located in a position on an imaginary line extending from an axis of the guide member; and
a light beam radiating member radiating a light beam traveling along the imaginary line extending from the axis of the guide member.

2. A drill guide for an anterior cruciate ligament reconstruction operation according to claim 1, comprising:
a pointing member supporting arm supporting the pointing member; and
a mirror provided at a tip part of the pointing member supporting arm.

3. A drill guide for an anterior cruciate ligament reconstruction operation according to claim 2, wherein the pointing member supporting arm includes a water supplying part.
